(19) United States
(12) Patent Application Publication
(10) Pub. No.: US 2020/0093388 A1
(43) Pub. Date: Mar. 26, 2020

(54) Systems and Methods for Processing and Presenting Arrhythmia Information for the Identification of Neurological Disease

(71) Applicant: Braemar Manufacturing, LLC, Eagan, MN (US)

(72) Inventors: Radouane Bouguerra, San Diego, CA (US); Wayne Derkac, Walloon Lake, MI (US); JR Finkelmeier, Malvern, PA (US); David Shanes, Pacheco, CA (US)

(21) Appl. No.: 16/137,910

(22) Filed: Sep. 21, 2018

(51) Int. Cl. A61B 5/044; G16H 15/00; G16H 10/65; A61B 5/046; A61B 5/0468

(52) U.S. Cl. CPC A61B 5/044; G16H 15/00; A61B 5/0468; A61B 5/046; G16H 10/65

(57) ABSTRACT

A system for reporting cardiologic data includes a patient-portable monitoring device and circuitry. The monitoring device is configured to detect electrocardiogram (ECG) data and patient-initiated event data. The circuitry is configured to receive the ECG data and the patient-initiated event data; detect atrial fibrillation (AF) events in the ECG data; calculate the duration of each AF event by subtracting the respective start time from the respective stop time of each AF event; compare the duration of each AF event to a first duration threshold; store each AF event having a duration exceeding the first duration threshold; calculate a monitoring time period duration by subtracting the monitoring start time from the monitoring stop time; calculate, based on the stored AF events, AF burden; and output a graphical presentation of the patient-initiated event data, AF burden, and stored AF events. The first duration threshold is less than 30 seconds.

### BACKGROUND

Various aspects of the functioning of the heart can be tracked by monitoring the heart's electrical activity. This electrical activity is commonly recorded as an electrocardiogram (ECG) using electrodes placed on a body surface. Anomalous electrical activity recorded in the ECG reports can be indicative of disease states or other physiological conditions, such as atrial fibrillation (AF). AF involves the loss of synchrony between the upper chambers of the heart (atria) and the lower chambers of the heart (ventricles). In complex AF, long-lived wave-like oscillations (wavelets) of depolarization travel along circular paths in the atria. This can lead to irregular ventricular contraction as well as blood stagnation. AF has been associated with cardiac disease as well as stroke.

ECG data is commonly presented in ECG reports designed for cardiologists. For instance, an episode of AF is conventionally defined as an event lasting more than 30 seconds. Detecting short duration (e.g., <30 seconds) AF events is believed by some to increase the rate of false positives for cardiac disease compared to detecting only longer duration (e.g., >30 seconds) AF events. Such false positives, if not caught by a physician, can cause unnecessary or unhelpful medical treatment. See Frank Bogun et al., Misdiagnosis of atrial fibrillation and its clinical consequences, 117 THE AMERICAN JOURNAL OF MEDICINE 636-642 (2004). Accordingly, conventional ECG reports do not typically include information regarding AF events under 30 seconds in duration. These short-duration AF events may be relevant to diagnosing certain neurological diseases even if they are less relevant to cardiac disease. For example, certain short duration AF can indicate an increased risk of stroke but not a significantly increased risk of cardiac disease (e.g., congestive heart failure). This may be because even short duration AF indicates changes in the atrium that can later cause stroke. For instance, short duration AF may be indicative of atrial cardiomyopathy, impaired atrial conduction, or other diseases of the atrial wall which could change clotting mechanisms in the heart and cause stroke.

While ECG data is typically used by cardiologists to diagnose cardiac disease, it can also be used by neurologists to detect AF and other cardiac arrhythmias associated with neurological disease. However, conventional ECG reports, which are designed for interpretation by cardiologists, can be difficult for neurologists to use for diagnosing neurological disease. For example, ECG reports commonly include traces of the raw ECG data measured during arrhythmia events, which can be difficult to read for neurologists, who are not cardiac specialists. Furthermore, neurologists often need to analyze additional data, such as short-duration AF events, and broad trends may not be included in conventional ECG reports.

### SUMMARY

Systems, methods, and devices for processing and selectively presenting cardiac information to a medical practitioner are presented. The systems include circuitry that processes ECG data and outputs a graphical presentation of a set of cardiac information suited to the diagnosis of neurological disease. In particular, according to an embodiment, the report output by the circuitry includes a graphical presentation of AF burden, detected AF events, and patient-initiated events. The graphically presented AF events may include AF events having a short duration (e.g., <30 seconds, <25 seconds, <20 seconds, <15 seconds, <10 seconds or any other suitable duration).

The report may also include a graphical presentation of patient-initiated event data. The system may register one or more patient-initiated events when the system receives an indication that a patient has manually indicated that the patient has experienced a symptom, including symptoms that might be neurally mediated. The system may determine whether the one or more patient-initiated events are concurrent with detected cardiac arrhythmia. By determining whether the one or more patient-initiated events are concurrent with detected cardiac arrhythmia, the system may distinguish between neurally mediated disease and cardiac disease. For example, fainting (syncope) can be neurally mediated or caused by cardiac arrhythmia, such as a cardiac pause event or a ventricular fibrillation (VF) event. Cardiac pause (also known as sinoatrial arrest, sinus arrest, or sinus pause) occurs when the sinoatrial node of the heart transiently ceases to generate the electrical impulses that normally stimulate the cardiac tissues to contract. Ventricular fibrillation occurs when the ventricles flutter erratically without coordination due at least in part to disorganized electrical activity in the ventricles, causing insufficient ejection of blood from the heart. If a patient-initiated syncope event coincides with a cardiac pause event, a ventricular fibrillation event, or any other serious cardiac arrhythmia, the system may determine that the syncope event was caused by cardiac arrhythmia and indicate that the syncope event is not primarily neurally mediated. In contrast, if a patient-initiated event indicates a syncope event that was not concurrent with cardiac arrhythmia, the system may indicate that syncope was neurally mediated.

In one aspect, a system for reporting cardiologic data includes a patient-portable monitoring device and circuitry. The patient-portable monitoring device is configured to detect electrocardiogram (ECG) data of a patient and patient-initiated event data. The ECG data includes a monitoring time period having a monitoring start time and a monitoring stop time. The circuitry is configured to receive the ECG data and the patient-initiated event data from the patient-portable monitoring device; detect atrial fibrillation (AF) events in the ECG data, wherein detecting AF events includes detecting a start time and a stop time for each detected AF event; calculate the duration of each AF event by subtracting the respective start time from the respective stop time of each AF event; compare the duration of each AF event to a first duration threshold; store each AF event having a duration exceeding the first duration threshold; calculate a monitoring time period duration by subtracting the monitoring start time from the monitoring stop time; calculate, based on the stored AF events, AF burden for the monitoring time period, wherein the AF burden for the monitoring time period is equal to a sum of the durations of each stored AF event occurring during the monitoring time period divided by the monitoring time period duration; and output a graphical presentation of the patient-initiated event data, AF burden, and stored AF events. The first duration threshold is less than 30 seconds.

In some implementations, the circuitry is further configured to exclude at least a portion of one or more ECG traces from the graphical presentation. In certain implementations, the monitoring time period is a first monitoring time period, the ECG data includes a second monitoring time period occurring, after the first monitoring time period, having a second monitoring start time and a second monitoring stop time. In such implementations, the circuitry is further configured to calculate the second monitoring time period duration by subtracting the second monitoring start time from the second monitoring stop time, and calculate, based on the stored AF events, AF burden for the second monitoring time period, the AF burden for the second monitoring time period equaling a sum of the durations of each stored AF event occurring during the second monitoring time period divided by the second monitoring time period duration. In such implementations, outputting the graphical presentation of AF burden includes graphically presenting a plot of AF burden over time, the plot including a first axis representing time, a second axis representing a level of AF burden, a graphical indication of the AF burden for the first monitoring time period, and a graphical indication of the AF burden of the second monitoring time period.

In some implementations, the second axis is scaled logarithmically. In certain implementations, outputting a pictographic presentation of the AF burden includes outputting a percentage representing the fraction of time spent in AF in a monitoring time period. In some implementations, outputting a graphical presentation of the AF events includes outputting a count of AF events. In certain implementations, the circuitry is further configured to present the respective duration of each of the graphically presented AF events. In some implementations, the circuitry is further configured to graphically present a plot of the duration of each of the presented AF events over time. In certain implementations, outputting a graphical presentation of the patient-initiated events includes outputting a count of the patient-initiated events. In some implementations, the circuitry is further configured to detect cardiac pause events in the ECG data and output a graphical presentation of the pause events. In certain implementations, outputting a graphical presentation of the pause events includes outputting a count of the pause events. In some implementations, the circuitry is further configured to output the patient-initiated event data, AF events, and pause events on a common time scale.

In certain implementations, the circuitry is further configured to detect ventricular fibrillation events in the ECG data; output a graphical presentation of the ventricular fibrillation events; determine a severity score of the detected ventricular fibrillation events; compare the respective severity score of each of the detected ventricular fibrillation events to a severity threshold; and exclude, from the graphical presentation, a ventricular fibrillation event having a severity score less than the severity threshold. In some implementations, the circuitry is further configured to compare the duration of each AF event to a second duration threshold and store a representation of each AF event having a duration less than the second duration threshold in a short duration AF database. In such implementations, the second duration threshold is greater than the first duration threshold. In certain implementations, the second duration threshold is 30 seconds. In some implementations, outputting the graphical presentation includes visually distinguishing, on the graphical presentation, the AF events stored in the short duration AF database from those AF events have a duration exceeding the first duration threshold and not represented in the short duration AF database.

In another aspect, a method for reporting cardiologic data includes receiving ECG data and patient-initiated event data from the patient-portable monitoring device, the ECG data including a monitoring time period comprising a monitoring start time and a monitoring stop time, the patient-initiated event data including a first patient-initiated event at a first time; detecting atrial fibrillation (AF) events in the ECG data, including detecting an AF start time and an AF stop time for each detected AF event; calculating the duration of each AF event by subtracting the respective AF start time from the respective AF stop time of each AF event; comparing the duration of each AF event to a first duration threshold; storing each AF event having a duration exceeding the first duration threshold; calculating a monitoring time period duration by subtracting the monitoring start time from the monitoring stop time; calculating, based on the stored AF events, AF burden for the monitoring time period, the AF burden for the monitoring time period equaling a sum of the durations of each stored AF event occurring during the monitoring time period divided by the monitoring time period duration; and outputting a graphical presentation of the patient-initiated event data, AF burden, and stored AF events. The first duration threshold is less than 30 seconds.

In some implementations, the method includes excluding at least a portion of one or more ECG traces from the graphical presentation. In certain implementations, the monitoring time period is a first monitoring time period, the ECG data includes a second monitoring time period occurring after the first monitoring time period and comprising a second monitoring start time and a second monitoring stop time, and the method also includes calculating the second monitoring time period duration by subtracting the second monitoring start time from the second monitoring stop time, and calculating, based on the stored AF events, AF burden for the second monitoring time period, the AF burden for the second monitoring time period equaling a sum of the durations of each stored AF event occurring during the second monitoring time period divided by the second monitoring time period duration, and outputting the graphical presentation of AF burden including graphically presenting a plot of AF burden over time. In such implementations, the plot includes a first axis representing time, a second axis representing a level of AF burden, a graphical indication of the AF burden for the first monitoring time period, and a graphical indication of the AF burden of the second monitoring time period. In some implementations, the second axis is based on a logarithmic scale.

In certain implementations, the method also includes comparing the duration of each AF event to a second duration threshold, the second duration threshold being greater than the first duration threshold; storing a representation of each AF event having a duration less than the second duration threshold in a short duration AF database; and visually distinguishing the AF events represented in the short duration AF database from AF events not represented in the short duration AF database. In some implementations, the method also includes detecting cardiac pause events in the ECG data and outputting a graphical presentation of the pause events. In certain implementations, the method also includes receiving a proximity threshold time; for each pause event, determining whether a start time of the pause event is greater than the sum of the first time of the first patient-initiated event and the proximity threshold time; if the start time of the pause is event is not greater than the sum of the first time and the proximity threshold time, determining whether an end time of the pause event is less than the first time minus the proximity threshold time; and if the start time of the pause is event is not greater than the sum of the first time and the proximity threshold time, and if the end time of the pause event is not less than the first time minus proximity threshold time, indicating in the graphical presentation that the patient-initiated event and the pause event are concurrent.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

### BRIEF DESCRIPTION OF THE DRAWINGS

FIG. 1 shows, according to an illustrative implementation, a system for reporting information related to cardiac events.

FIG. 2 shows, according to an illustrative implementation, a graphical presentation of information related to cardiac events.

FIG. 3 shows, according to an illustrative implementation, a graphical presentation of information related to cardiac events including AF burden over time.

FIG. 4 shows, according to an illustrative implementation, a timeline representing AF events taking place over a period of time.

FIG. 5 shows, according to an illustrative implementation, a graph representing the frequency and duration of AF events.

FIG. 6 shows, according to an illustrative implementation, a graph representing AF events and the associated AF burden.

FIG. 7 shows, according to an illustrative implementation, a graph representing the incidence of cardiac events taking place over a period of time.

FIG. 8 shows, according to an illustrative implementation, a graph representing the frequency of cardiac events taking place over a period of time.

FIG. 9 shows, according to an illustrative implementation, a graph representing the incidence and frequency of cardiac events taking place during a period of time.

FIG. 10 shows, according to an illustrative implementation, a flow chart of a method for reporting cardiologic data.

FIG. 11 shows, according to an illustrative implementation, a flow chart of a method for reporting cardiologic data.

FIG. 12 shows, according to an illustrative implementation, a flow chart of a method for reporting cardiologic data, distinguishing between short duration and long duration events.

FIG. 13 shows, according to an illustrative implementation, a flow chart of a method for determining whether patient-initiated events are concurrent with automatically detected events.

### DETAILED DESCRIPTION

Systems, methods, and devices for processing and selectively presenting cardiac information to a medical practitioner are presented. The systems include circuitry that processes ECG data and outputs a graphical presentation of a set of cardiac information suited to the diagnosis of neurological disease. In particular, the report output by the circuitry includes a graphical presentation of AF burden, detected AF events, and patient-initiated events. The graphically presented AF events may include AF events having a short duration (e.g., <30 seconds, <25 seconds, <20 seconds, <15 seconds, <10 seconds or any other suitable duration).

The report may also include a graphical presentation of patient-initiated event data. The system may register patient-initiated events when the system receives an indication that a patient has manually indicated that the patient has experienced a symptom, including a symptom that might be neurally mediated. The system may determine whether the patient-initiated events are concurrent with detected cardiac arrhythmia. By determining whether patient-initiated events are concurrent with detected cardiac arrhythmia, the system may distinguish between neurally mediated disease and cardiac disease. For example, fainting (syncope) can be neurally mediated or caused by cardiac arrhythmia, such as a cardiac pause event or a ventricular fibrillation event. Cardiac pause (also known as sinoatrial arrest, sinus arrest, or sinus pause) occurs when the sinoatrial node of the heart transiently ceases to generate the electrical impulses that normally stimulate the cardiac tissues to contract. Ventricular fibrillation occurs when the ventricles flutter erratically without coordination due to disorganized electrical activity in the ventricles, causing insufficient ejection of blood from the heart. If a patient-initiated syncope event coincides with a cardiac pause event, a ventricular fibrillation event, or any other serious cardiac arrhythmia, the system may determine that the syncope event was caused by cardiac arrhythmia and indicate that the syncope event is not primarily neurally mediated. In contrast, if a patient-initiated event indicated a syncope event that was not concurrent with cardiac arrhythmia, the system may indicate that syncope was neurally mediated.

As used herein, “graphically presenting” data includes, but is not limited to, displaying data by means of a marker, chart, graph, plot, pictograph, numerals, symbols or any other suitable graphical representation and combination thereof. Pictographic presentation includes a type of graphic presentation using one or more pictorial symbols. In other words, pictographic presentation includes showing data using images or symbols.

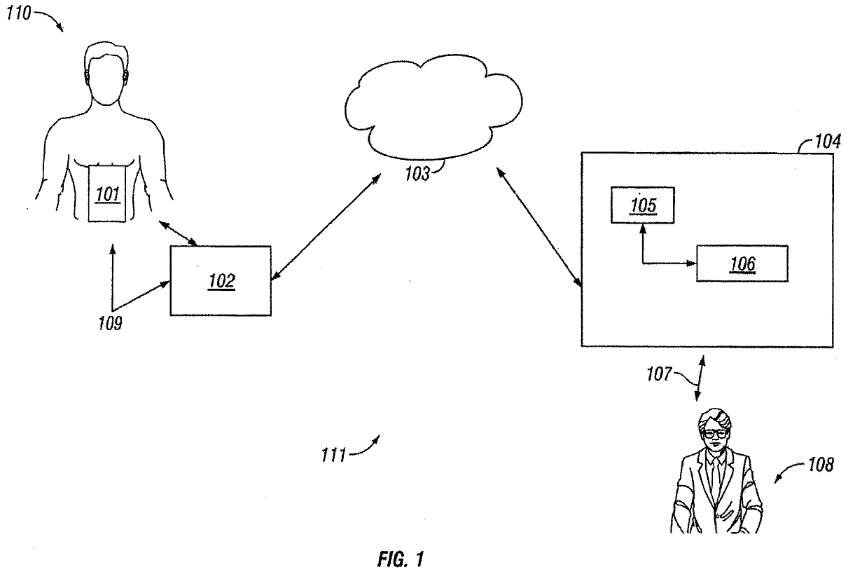

FIG. 1 illustrates, according to an exemplary embodiment, a system 111 for reporting information related to cardiac events, such as atrial fibrillation (AF) events and AF burden, which is the amount of time spent in AF or the proportion of a monitoring period (such as, for example, a day) spent in AF. The system 111 includes a monitoring system 109, a communication network 103, a monitoring center 104, and a transmission path 107. The system 111 can be used by a physician, such as a neurologist, or other healthcare provider 108. The monitoring system 109 can communicate (via the devices 101 and 102) electrocardiogram (ECG) data, patient-initiated event data, and other data to the monitoring center 104. The monitoring system 109 includes, in some implementations, an implantable medical device (IMD), such as an implantable cardiac defibrillator and an associated transceiver or pacemaker and an associated transceiver. The monitoring system 109 may also include the monitoring device 101 that is worn by the patient 110 or that is patient-portable. Further, the monitoring system 109 can include the monitor processing device 102 that can send standard physiological data (received from monitoring device 101) to the monitoring center 104. In some implementations, the monitor processing device 102 automatically detects arrhythmia events, including start and stop times for each event. The detected events may include AF events, ventricular fibrillation events, pause events, and/or any other cardiac arrhythmia event. The monitoring system 109 may also record a type of cardiac event referred to herein as a “patient-initiated event”, which is triggered in response to a deliberate action of the patient, (e.g., in response to the patient 110 pressing a button on the device 101), and transmit the patient-initiated event data to the monitoring center 104. In one implementation, the devices 101 and 102 are integrated into a single device. Moreover, the system 109 can be implemented using, for example, the CardioNet Mobile Cardiac Outpatient Telemetry (MCOT) device, which is commercially available and provided by CardioNet, Inc. of Malvern, Pa.

The monitor processing device 102 can transmit physiological data, including data related to arrhythmia events and patient-initiated events, through the communication network 103, which can be a local area network (LAN), a landline telephone network, a wireless network, a satellite communication network, or other suitable network to facilitate two-way communication with the monitoring center 104. Advantageously, the monitoring center 104 can be located in the same location (e.g., in the same room or building) as the monitoring system 109, or at some remote location.

The monitoring center 104 can include the monitoring (or display) station 105 and the processing system 106. In some implementations, the processing system 106 automatically identifies arrhythmia events, such as AF and ventricular fibrillation. In certain implementations, a cardiovascular technician (CVT) can use the monitoring station 105 to evaluate, or validate, physiological data received from the monitoring system 109, identifying and reporting, among other things, arrhythmia events (such as AF events). In some implementations, the processing system 106 receives and analyzes human-assessed arrhythmia event data from the CVT and/or automatically detected arrhythmia event data reported by the monitoring system 109 to determine whether to generate a report or graphical presentation related to these events. In certain circumstances, the processing system 106 will send the report related to arrhythmia and/or patient-initiated events, for example, to a neurologist, physician, or other health care provider 108 via the transmission path 107—which may be part of the network 103. The report may be a static presentation or programmatically responsive to user input, such as may be provided through a web interface, keyboard, mouse, or any other suitable means, to allow a medical practitioner to specify time ranges and alternate displays. The monitoring center 104 may receive user input or queries, for example through the monitoring station 105 or over the transmission path 107, to specify a time or date range or an alternate report format, for example, a format that adds one or more sections to the report or changes the contents of the one or more sections. In certain implementations, the processing system 106 determines whether the report or graphical presentation omits one or more ECG traces. In some implementations, the one or more ECG traces may be available upon request by the user. For example, a user may be able to toggle on or off the display of ECG traces or select a section of the report to see ECG traces corresponding to the events.

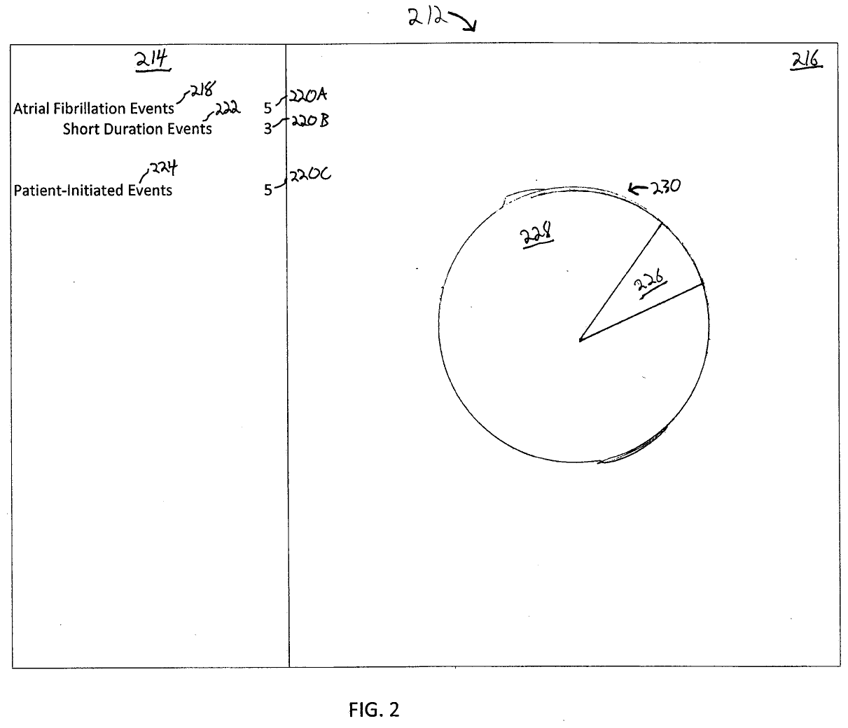

FIG. 2 shows, according to an illustrative implementation, a report 212 of information related to cardiac events. The report 212 graphically presents the data collected by a monitoring system (e.g., monitoring system 109) in a manner that indicates a risk of neurological disease or impairment, which is referred to herein as “neurological risk.” The report includes a first report section 214 and a second report section 216. The report section 214 includes an atrial fibrillation section 218, a count of AF events 220A, a short duration atrial fibrillation section 222, a count of short duration AF events 220B, a patient-initiated event section 224, and a count of patient-initiated events 220C. The second report section 216 includes a chart 230 representing AF burden. The chart 230 includes a sector 226 representing the proportion of time a patient was in AF and a sector 228 representing the proportion of time the patient was not in AF. The report 212 in FIG. 2 is provided for illustrative purposes only, and any one of these report sections or counts can be omitted or rearranged, and additional elements added as needed, as will be understood by those of ordinary skill in the art.

The report section 214 of the report 212 lists types of cardiac arrhythmia events associated with a risk of neurological disease (risk events) and incident counts for each type. One type of event listed by the report section 214 is AF events in the atrial fibrillation section 218. The duration of AF events in the atrial fibrillation section 218 may vary and the report can include events detected and reported by a CVT, a processing system, and/or a monitoring system, any of which may be capable of detecting AF events that are 30 seconds or less in duration (e.g., <30 seconds, <25 seconds, <20 seconds, <15 seconds, <10 seconds or any other suitable duration). The incident count 220A shows the number of atrial fibrillation events that are included in the report. Some AF events in atrial fibrillation section 218 are indicated as being short duration events 222. The short duration events 222 are AF events that are longer than a first duration threshold, but shorter than a second duration threshold. For instance, the first duration threshold may be less than 30 seconds, and the second duration may be 30 seconds or any other suitable time period for determining short-duration AF events (e.g., 29 seconds, 28 seconds, 27 seconds, 26 seconds, 25 seconds, 20 seconds, 15 seconds, 10 seconds, <10 seconds, or any other suitable duration). In certain implementations, the first threshold is substantially less than 30 seconds. In some implementations, the first threshold is 29 seconds, 28 seconds, 27 seconds, 26 seconds, 25 seconds, 20 seconds, 15 seconds, 10 seconds, or <10 seconds. In some implementations, the first duration threshold, the second duration threshold, or both are based on a number of heart beats rather than a number of seconds. For example, the first duration threshold may be 45 beats, 43 beats, 40 beats, 35 beats, 30 beats, 25 beats, 20 beats, 15 beats, 10 beats, 5 beats, 4 beats, 3 beats, 2 beats, 1 beat, or any other suitable duration. The second duration threshold can be greater than the first duration threshold by 1 beat, 2 beat, 3 beats, 4 beats, 5 beats, 6 beats, 7 beats, 8 beats, 9 beats, 10 beats, 15 beats, 20 beats, 25 beats, 30 beats, 35 beats, 40 beats, 43 beats, 45 beats, 50 beats, 60 beats, 70 beats, 100 beats, or any other suitable duration. In some implementations, the first duration threshold, the second duration threshold, or both are fixed or otherwise predetermined. In certain implementations, the first duration threshold, the second duration threshold, or both are variable and may be selected or determined by an end user, such as the healthcare provider (such as, for example, a physician) 108.

The thresholds may be varied to detect ECG arrhythmia events with a desired sensitivity or specificity (e.g., based on the MIT-BIH Arrhythmia Database, described in more detail in a subsequent paragraph). For example, the first duration threshold can be set to vary such that the specificity is high (e.g., 55%, 60%, 70% 80%, 85%, 90%, 91%, 92%, 93%, 94%, 95%, 96%, 97%, 98%, 99%, 99.5%, 99.9%, 99.99%) and the sensitivity is high (e.g., 55%, 60%, 70% 80%, 85%, 90%, 91%, 92%, 93%, 94%, 95%, 96%, 97%, 98%, 99%, 99.5%, 99.9%, 99.99%). In some implementations, short duration AF events 222 are defined with reference to a sensitivity threshold and a specificity threshold rather than duration thresholds. For example, a short duration AF event may be an event detected using a method having a sensitivity and a specificity exceeding a first set of thresholds (e.g., 90% specificity and 90% sensitivity), but not exceeding a second set of thresholds (e.g. 95% specificity and 95% sensitivity).

The sensitivity or specificity of an ECG detection process can be determined by processing an ECG test data set which has pre-identified arrhythmia events. For example, the Massachusetts Institute of Technology and the Beth Israel Hospital (now the Beth Israel Deaconess Medical Center) have published an ECG arrhythmia database (the MIT-BIH Arrhythmia Database) that has been one of the standards for determining sensitivity and specificity of ECG detection processes for years. See George B. Moody & The impact of the MIT-BIH Arrhythmia Database, 20 IEEE ENGINEERING IN MEDICINE AND BIOLOGY MAGAZINE 45-50 (2001). The MIT-BIH Arrhythmia Database contains 48 half-hour excerpts of two-channel ambulatory ECG recordings, obtained from 47 subjects studied by the BIH Arrhythmia Laboratory between 1975 and 1979. The recordings were digitized at 360 samples per second per channel with 11-bit resolution over a 10 mV range. Two or more cardiologists annotated the set of cardiac arrhythmia events that would serve as the standard (later called the “pre-identified events” or “annotated events”). The cardiologists decided on the pre-identified events by independently annotating each record in the MIT-BIH Arrhythmia Database and resolving discrepancies by mutual agreement. The MIT-BIH Arrhythmia Database includes approximately 110,000 pre-identified and cardiologist-confirmed events. See A. L. Goldberger, et al., PhysioBank, PhysioToolkit, and PhysioNet: Components of a New Research Resource for Complex Physiologic Signals, 101 CIRCULATION, 215-20 (2000) <circ.ahajournals.org/content/101/23/e215.full>. The MIT-BIH Arrhythmia Database is available for download at: <physionet.org/physiobank/database/mitdb/>. The sensitivity of an ECG detection process can be determined by processing the MIT-BIH Arrhythmia Database and then determining the percentage of pre-identified events that were correctly identified as arrhythmia by the ECG detection process. The specificity of the ECG detection process can be determined by processing the MIT-BIH Arrhythmia Database and then determining the percentage of normal heart beats (heart beats that were not in the set of pre-identified events) that were correctly identified by the ECG detection process as not cardiac arrhythmia. The sensitivity and specificity determinations can be made in this manner for any first duration threshold or any second duration threshold. Therefore, each duration threshold can be associated with a sensitivity measure and a specificity measure using the MIT-BIH Arrhythmia Database as the standard. Thus, in some implementations, the first duration threshold and/or the second duration threshold is set based on its sensitivity and/or specificity as determined with reference to the MIT-BIH Arrhythmia Database.

The short duration events 222 are associated with an incident counter 220B that indicates how many of the events counted in incident counter 220A qualify as short duration AF events and are included in the short duration atrial fibrillation section 222. This can allow short duration events to be given greater or lesser importance by the medical practitioner depending on the disease being diagnosed. For example, short duration AF may be more relevant to diagnosing neurological disease than cardiac disease because certain short duration AF may be associated with an increased risk of stroke but not with a significantly increased risk of cardiac disease (e.g., congestive heart failure). This may be because short duration AF indicates changes in the atrium that can later cause stroke. For instance, short duration AF may be indicative of atrial cardiomyopathy, impaired atrial conduction, or other diseases of the atrial wall which could change clotting mechanisms in the heart and cause stroke.

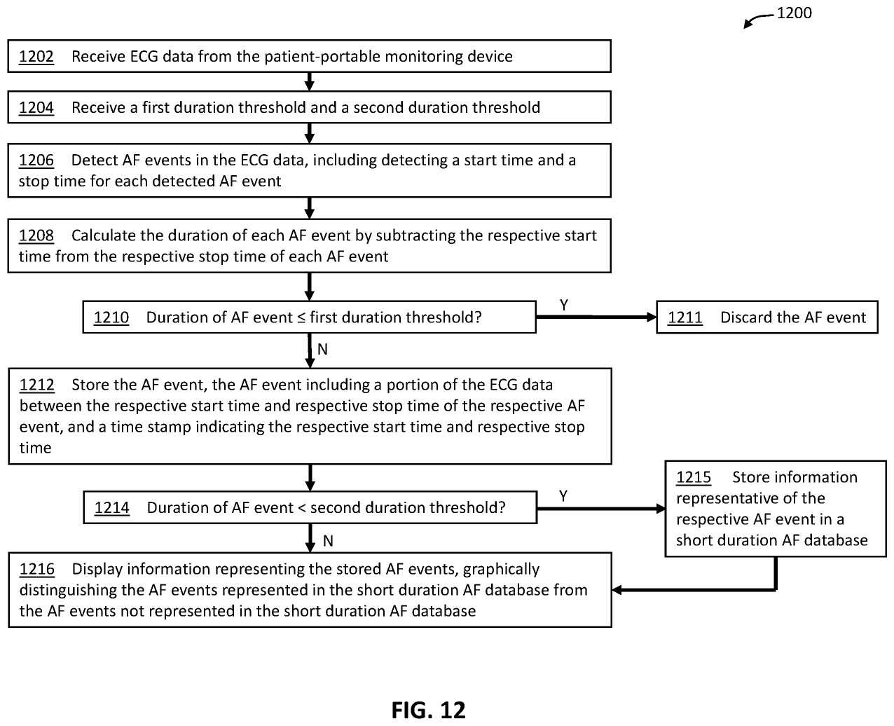

Another type of risk event listed in report section 214 are patient-initiated events, which are shown in the patient-initiated events section 224. These are events initiated or recorded by a patient (e.g., the patient 110) using an input to the monitoring system (e.g., the monitoring system 109). Patient-initiated events allow the report 212 to indicate moments when the patient subjectively experiences possible symptoms of cardiac distress, syncope (such as partial or complete loss of consciousness), or any other unusual or abnormal condition. The patient-initiated events 224 may not be mutually exclusive to the other risk events. For example, the report 212 may indicate that the patient indicated a patient-initiated event at the same time that an AF event was automatically detected by the monitoring system (e.g., monitoring system 109). The incident count 220C may show the number of patient-initiated events included in the report 212. By determining whether the patient-initiated events are concurrent with detected cardiac arrhythmia, the report 212 may distinguish between events that may be indicative of a neurally mediated disease and events that may be indicative of a cardiac disease (e.g., neurally mediated syncope versus cardiac syncope). As will be discussed in relation to FIG. 12, if a patient-initiated syncope event coincides with, or nearly coincides with, a cardiac pause event, a ventricular fibrillation event, or any other serious cardiac arrhythmia, the report 212 may indicate that the syncope is not primarily neurally mediated. In contrast, if a patient-initiated event indicates a syncope event that was not concurrent or close in time with cardiac arrhythmia, the report 212 may indicate that the syncope is neurally mediated.

The second report section 216 includes the chart 230, which graphically presents the AF burden experienced by the patient (e.g., the patient 110). The sector 226 shows the total duration of AF events 218 as a proportion of the time period covered by the report 212. The sector 228 shows the total duration of normal atrial rhythm as a proportion of the time period covered by the report. Although the chart 230 is represented as a pie chart in FIG. 2, in some implementations the AF burden is depicted as a bar chart, a line graph, a percentage, or in any other suitable format.

In the implementation shown in FIG. 2, the report 212 does not include visual representations of raw ECG traces. Instead, the report 212 includes results derived from analysis of the raw ECG data. This presentation may facilitate review of the report 212 by neurologists or other medical practitioners who are not specialized in cardiology. In some implementations, the report 212 may include visual representations of one or more raw ECG traces. In certain implementations, ECG traces are available upon request by the user. For example, a user may be able to toggle on or off the display of ECG traces or select a section of the report to see the one or more ECG traces corresponding to the events.

The ECG data used to generate the information in the report 212 may be obtained from the monitoring system 109 of FIG. 1 or any other suitable processing system. The report may be derived from data recorded by a monitoring system in a fixed time period (e.g., 24 hours), a time period specified by a medical professional, or a time period determined in any other suitable manner. In some implementations, the report is derived from all the recorded ECG data.

The selection of events displayed in the report 212 may be tailored to detection of neurological disease. AF (including short duration AF) and patient-initiated events can be of particular importance to diagnosing neurological disease, such as stroke or neurally mediated syncope. Therefore, inclusion of the atrial fibrillation section 218, short duration atrial fibrillation section 222, and a patient-initiated event section 224 in the report may aid a neurologist or other medical practitioners in diagnosing neurological disease. The short duration atrial fibrillation section 222 may be of unique interest to neurologists because short duration atrial fibrillation may be more indicative of a risk of a neurological disease (e.g., stroke) than a risk of a cardiac disease.

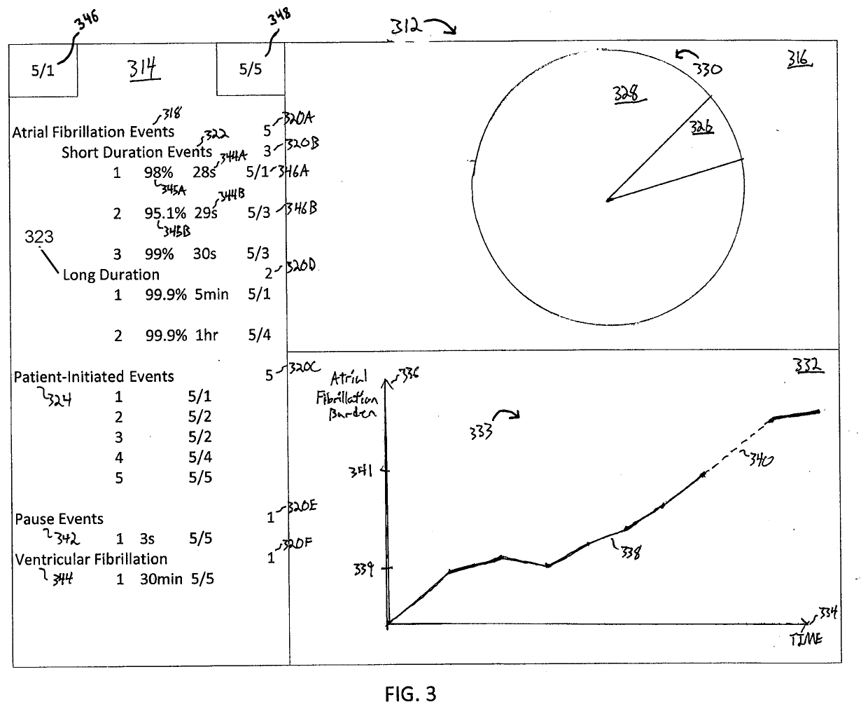

FIG. 3 shows a more detailed view of a report 312 of information related to cardiac events. The report 312, like the report 212 of FIG. 2, graphically presents ECG data in a manner that may facilitate the diagnosis of neurological risk, cardiac risk, or both. The report 312 includes a first report section 314, a second report section 316, and a third report section 332. The first report section 314 includes an atrial fibrillation section 318, a count of AF events 320A, a short duration atrial fibrillation section 322, a short duration atrial fibrillation section 323, a count of short duration AF events 320B, a specificity 345A of an AF event diagnosis, a specificity 345B of an AF event diagnosis, a duration 344A of an AF event, a duration 344B of an AF event, a timestamp 346A of an AF event, a timestamp 346B of an AF event, a count of long duration AF events 320D, a patient-initiated event section 324, a count of patient-initiated events 320C, a pause event section 342, a count of pause events 320E, a ventricular fibrillation event section 344, a count of ventricular fibrillation events 320F, a start date 346, and an end date 348. The report section 316 includes a chart 330 representing AF burden. The chart 330 includes a sector 326 representing the proportion of time a patient was in AF and a sector 328 representing the proportion of time the patient was not in AF. The third report section 332 includes a chart 333 representing AF burden over time. The chart 333 includes an axis 334 representing time, an axis 336 representing AF burden, a line 338 representing AF burden, a dashed line segment 340 depicting interpolation in a period during which no ECG measurement was recorded, a first threshold 339, and a second threshold 341. The report 312 is sectioned similarly to the report 212 of FIG. 2, except with the addition of report section 332. The report 312 in FIG. 3 is provided for illustrative purposes only, and any one of these report sections or counts can be omitted or rearranged, and additional elements added as needed, as will be understood by those of ordinary skill in the art.

The report section 314 of the report 312 lists types of risk events and incident counts for each type. One type of risk event listed in the report section 314 is the pause events in pause event section 342, which represent periods in which the heart does not generate the requisite electrical impulses for at least a brief period (e.g., 2 seconds or more). Pause events can cause a temporary lack of blood flow that may increase the likelihood of the formation of blood clots (thrombi) and may lead to ischemic stroke. Pause events can also be one of multiple possible causes of syncope events. The inclusion of pause events in an ECG report may differentiate syncope caused by pause events from syncope caused by other cardiac conditions (e.g., transient ischemic attack) or a neurological condition (e.g., neurally mediated syncope). The report section 314 also lists ventricular fibrillation (VF) events in the VF events section 344. The processing system generating the report 312 (e.g., the processing system 106) may be configured to selectively include only level 1 events, which are events severe enough to cause death or serious disability. In some implementations, the processing system also reports level 2 events, which are events that do not require medical intervention to prevent death or serious disability. Ventricular tachycardia events or ventricular fibrillation events may be less likely to be relevant to stroke, so they may be assigned a lower priority than AF and other incidents if the VF events are not particularly serious (e.g., not level 1). In some implementations, to determine whether a non-AF cardiac arrhythmia event (e.g., ventricular tachycardia or ventricular fibrillation) is a level 1 event, a severity score is assigned to each detected non-AF event. The severity score can be calculated based on duration of the event, the frequency of ventricular depolarization during the event, or any other suitable metric. For example, the severity score may be the number of consecutive beats during which the non-AF event occurred. The severity score of each non-AF event is compared to a severity threshold. For example, the severity threshold may be 5 beats. If the non-AF event has a severity score greater than the severity threshold (e.g., the event lasted for longer than 5 beats), the non-AF event is a level 1 event and is included in the report 312. If a non-AF event has a severity below the severity threshold, then that non-AF event is not included in the report 312.

The first report section 314 also lists the reported events in individual detail and in chronological order. In some implementations, the events may be listed in another order, such as in order of decreasing severity or decreasing duration. Short duration AF events listed in the short duration atrial fibrillation section 322, for example, are listed individually with each event having a listed duration, for example 344A and 344B, and a respective timestamp or date of incidence—for example, 346A and 346B. In some implementations, the report 312 allows a user to select a listed date, such as the timestamp 346B, to specify a time and/or date range for the data used to generate the report 312 or a specific report section. The more detailed listing of event information can aid in diagnosis and eliminate the need for the original ECG trace by presenting the information that is relevant to neurological diagnosis. The report section 314 also includes a start date 346 and an end date 348 to allow a user (e.g., the neurologist or other healthcare provider 108) to understand and specify the date range in which the listed events took place. In some implementations, a user may use input or queries, for example through a monitoring station or over a transmission path, to specify a date range or an alternate report format. For example, a user may request a format that adds sections to the report or changes the contents of the sections.

The report section 314 also shows the specificity associated with the detected AF events 318. ECG monitoring systems generally must trade off sensitivity to AF (the true positive rate) with specificity to AF (the true negative rate). In other words, increasing the rate at which a monitoring system correctly identifies AF as AF may also increase the rate at which the monitoring system incorrectly identifies normal heart beats as AF. Reducing the minimum detectable AF event duration requires an increase in sensitivity to AF. This will reduce the specificity of AF detection, or, in other words, increase the rate of false positives. In some implementations, the processing system generating the report 312 will require an AF event of sufficient duration (e.g., 5 minutes) before reporting an AF event to have near certainty in the determination. In certain implementations, the processing system may have reduced specificity when it is configured to detect AF of a shorter duration. A patient may experience short duration AF (e.g., <30 seconds) for an extended period of time (e.g., a week, a month, several months, a year, or years) before experiencing longer AF events (e.g., >30 seconds, >5 minutes, >10 minutes). Thus, detecting the shorter duration AF may help to detect the disease in an earlier stage in the disease process at which point changes in the atrial tissue (substrate) are more reversible. A monitoring system may also detect an event lasting for a period of time that ends before the determination can be reached—for example, an event that lasts 5 seconds. In some implementations, the report 312 only includes events that exceed a specificity threshold and/or a duration threshold, which may be configurable by the user. The processing system generating the report 312 may detect more events than are displayed. For example, in certain implementations the processing system discards or withholds detected events that fail to meet a specificity and/or duration threshold.

The report section 332 displays the AF burden experienced by the patient 110 as a trend over time. The axis 334 represents time. The scale of the axis 334 may be determined by the date range specified by the start date 346 and the end date 348. In some implementations, the axis 334 scales independently of the other date ranges used in the report. For example, if the user desires to see a month of trend data but only the events of the week with the highest AF burden, the report section 332 may represent the events of the week with the highest AF burden.

The axis 336 represents AF burden. In some implementations, the axis 336 is presented in a logarithmic scale. The trend line 338 represents the trend in AF burden data. The dashed line segment 340 shows interpolation in a period during which no ECG measurement was recorded. In certain implementations, the trend line 338 would extend to zero or omit a segment during a period with no measured data or during a period with measured data indicating that a patient experienced no AF events. In some implementations, the trend line 338 is generated by linear interpolation such that it directly connects points representing the AF burden over a respective period of time. In certain implementations, the trend line 338 is generated by statistical regression over the points representing AF burden. In some implementations, the trend line 338 represents a moving average of AF burden or any other suitable means of presenting a trend to the user. In some implementations, the chart 333 includes a threshold 339 and a threshold 341. The threshold 339 may indicate a minimum amount of AF burden that is shown in the report 312 or considered clinically significant. In some implementations, the threshold 341 is used to show a minimum amount of AF burden that is shown or considered clinically significant. The thresholds 339 and 341 may also be used to mark levels of AF burden associated with the patient's history. In some implementations, the user may select whether to include AF events based on the duration of the events. For example, the neurologist may exclude AF events that do not exceed a set duration (e.g., 5 seconds, 10 seconds, 15 seconds, 20 seconds, 21 seconds, 22 seconds, 23 seconds, 24 seconds, 25 seconds, 26 seconds, 27 seconds, 28 seconds, 29 seconds, 30 seconds, 1 minute, 3 minutes, 5 minutes, 10 minutes, 30 minutes, 1 hour, or any other suitable time period). The thresholds 339 and 341 may be used to indicate and specify the duration bounds. In some implementations the neurologist may select the short duration AF events section 322 to toggle whether short duration AF events are included in the generation of the chart 332.

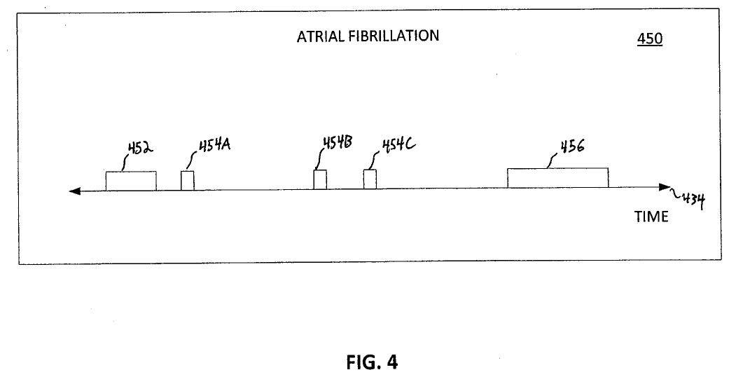

FIG. 4 shows, according to an illustrative implementation, a timeline graph 450 representing AF events taking place over a period of time. The timeline 450 can be included in the report 212, in the report 312, as a separate report, or in any other suitable manner. The timeline graph 450 graphically presents blocks representing long duration AF events 452 and 456 and blocks representing short duration AF events 454A, 454B, and 454C on a time axis 434. The blocks cover the duration of each AF event on the timeline. In some implementations, the timeline is divided into segments of time (e.g., 5 second, 15 second, 30 second, 1 minute, 5 minute, 15 minute, 30 minute, or 1 hour increments, or any other suitable increment) and the blocks snap to the boundaries of the bins. In some implementations, the blocks are placed as precisely as the display resolution will allow. The timeline graph 450 may be placed in a report section (e.g., 316) to replace a graph or graphical display, an additional report section, or to be displayed in any suitable manner.

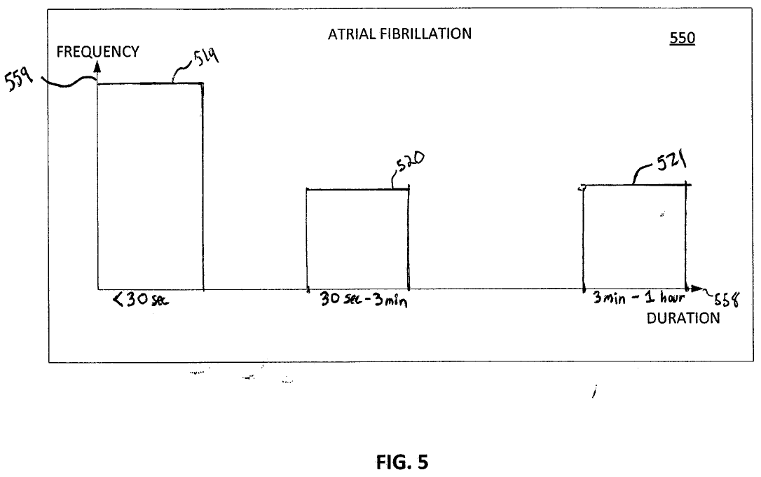

FIG. 5 shows, according to an illustrative implementation, a graph 550 representing the frequency and duration of AF events. The graph 550 includes a histogram that graphically presents counts of AF events that fall into several duration bins 519-521. The graph 550 includes a frequency axis 559, a duration axis 558, and the duration bins 519-521 that are presented on the duration axis 558. The position of bins 519-521 on the duration axis 558 indicates the duration of the events represented by each respective bin. The events represented by bin 519 have a duration of less than 30 seconds. The events represented by bin 520 have a duration of 30 seconds to 3 minutes. The events represented by bin 521 have a duration of 3 minutes to 1 hour. According to some implementations, the duration axis 558 has a logarithmic scale. According to other implementations, the duration axis has a linear scale. Any other suitable division, with greater, fewer, or the same number of bins, may be used. In some implementations the duration bins are specified by a neurologist, adjusted automatically based on the data, fixed ranges, or determined by any other suitable means. The graph 550 may be placed in a report section (e.g., section 316 of report 312) in place of a graph or graphical display. In some implementations, the graph 550 is an additional report section in report 312. In certain implementations, the graph 550 is displayed in a separate report or in any suitable manner. The method of presentation of data shown in FIG. 5 visually indicates how AF events are distributed as a function of duration.

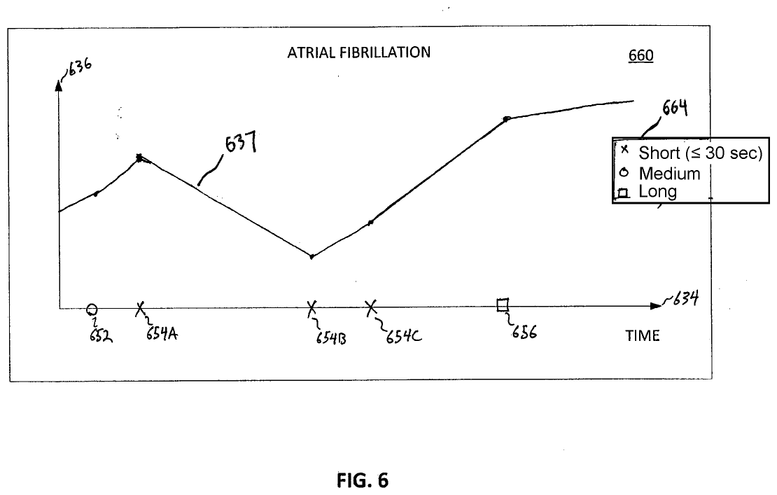

FIG. 6 shows, according to an illustrative implementation, a graph 660 including symbols 654A-C representing AF events and the associated AF burden 637. The graph 660 graphically presents AF events 652, 654A, 654B, 654C, and 656, using the symbols in legend 664, on a common time scale 634 with AF burden 637 plotted with respect to an AF burden axis 636. The AF events 652, 654A, 654B, 654C, and 656 are depicted using symbols that correspond to ranges of duration, as indicated in legend 664. As shown in graph 450 of FIG. 4, or graph 216 of FIG. 2, displaying the durations of the AF events (e.g., the AF event represented by symbol 656) that are responsible for the AF burden 637 allows a neurologist to determine whether the length or the frequency of AF events are driving AF burden. For example, the neurologist can distinguish between many short AF events versus fewer, longer AF events. Thus, this method of presentation can place AF burden trends and AF events on a common axis so that they can be reviewed together. In some implementations, the AF events may be shown using symbols colored according to a scale or gradient. For example, bright red symbols may indicate AF events having a duration of less than 10 seconds in duration or dark blue symbols may indicate AF events having a duration greater than 1 hour. In certain implementations, symbols scaled in length are used to show duration (e.g., events 454A and 456 in graph 450 of FIG. 4). The duration of AF events may be shown using any other suitable means.

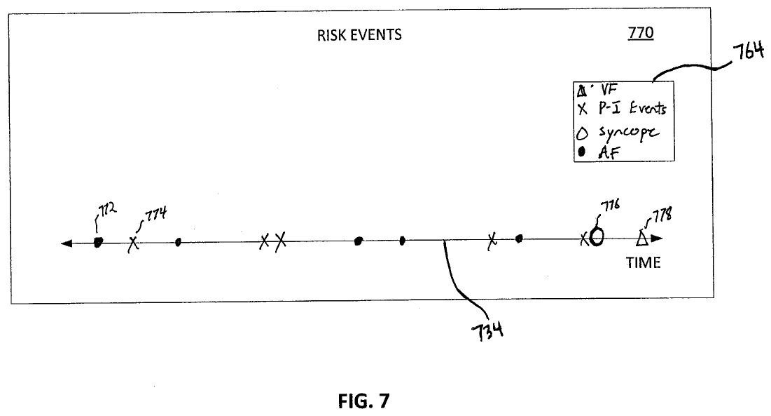

FIG. 7 shows, according to an illustrative implementation, a graph 770 representing the incidence of cardiac arrhythmia events taking place over a period of time. Graph 770 shows AF events, such as event 772, patient-initiated events, such as event 774, pause events, such as event 776, and VF events, such as event 778, on a time axis 734 using symbols indicated in legend 764. This presentation may be advantageous since it chronologically orders all the types of events on one scale, which makes it easier to determine whether patient-initiated events are concurrent with detected cardiac arrhythmia events and to use the information to distinguish between neurally mediated disease and cardiac disease (e.g., between neurally mediated syncope and cardiac syncope). For instance, if a patient-initiated syncope event coincides with a cardiac pause event, a level 1 ventricular fibrillation event, or any other serious cardiac arrhythmia, the graph 770 may indicate that a syncope is not primarily neurally mediated. In contrast, if a patient-initiated event indicated a syncope event that was not concurrent with cardiac arrhythmia, the graph 770 may indicate that the syncope was neurally mediated. In some implementations, the neurologist can specify which types of events to include by selecting event types in the legend 764 or any other suitable means.

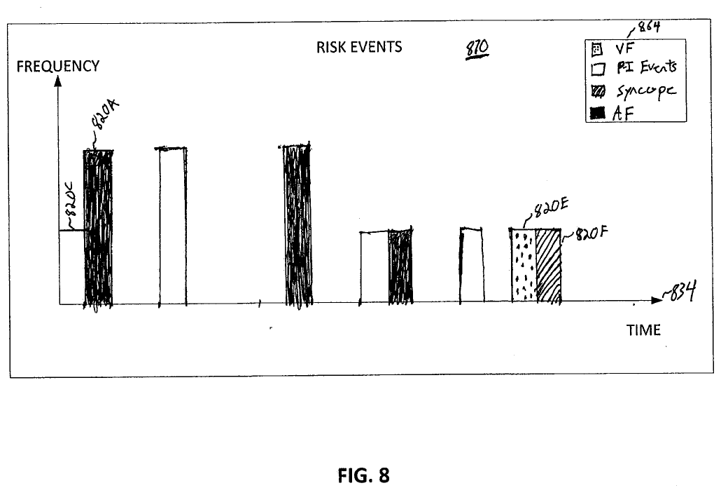

FIG. 8 shows, according to an illustrative implementation, a graph 870 including a legend 864 and bars 820A, 820C, 820E, and 820F representing the counts of AF events, counts of patient-initiated events, counts of VF events, and counts of syncope events, respectively, taking place over a period of time. The time axis 834 is divided into bins representing periods of time (e.g., days) which are divided again for bars representing the counts for each event type. In some implementations, the time period bins are specified by the neurologist, adjusted automatically based on the data, fixed ranges, or determined by any other suitable means. The presentation of graph 870 may indicate possible correlations in frequency across event types.

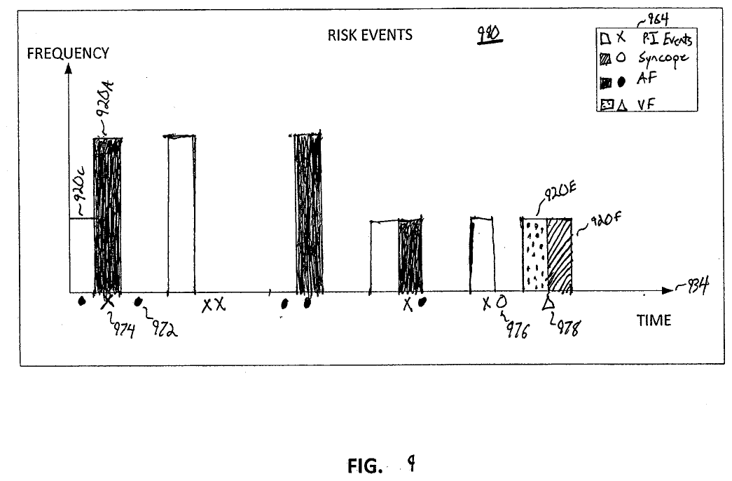

FIG. 9 shows, according to an illustrative implementation, a graph 980 representing the incidence and frequency of cardiac events taking place during a period of time. The graph 980 presents AF events (such as event 972), patient-initiated events (such as event 974), pause events (such as event 976), VF events (such as event 978), counts of AF events (e.g., 920A), counts of patient-initiated events (e.g., 920C), counts of VF events (e.g., 920E), and counts of syncope events (e.g. 920F) on a common time axis 934 using symbols identified in legend 964. Graph 980 superimposes the graphical presentations of graph 770 of FIG. 7 and graph 870 of FIG. 8 onto a common time axis 934. The format of graph 980 may indicate how events were distributed throughout a time period.

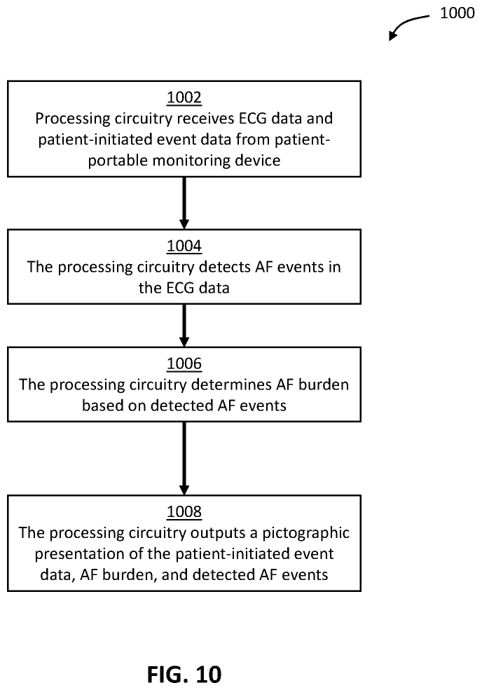

FIG. 10 shows a flow chart of a method 1000 for reporting cardiologic data according to certain implementations. In general, the method 1000 obtains ECG data from a monitoring system (e.g., monitoring system 109), analyzes the ECG data, and graphically presents—to a user (e.g., neurologist 108)—the data in, for example, the format of report 212 of FIG. 2, the format of report 312 of FIG. 3, or any other format suitable for neurological risk analysis. As shown, the method includes the processing circuitry receiving ECG and patient-initiated event data from a patient-portable monitoring device (step 1002). The processing circuitry detects AF events in the ECG data (step 1004), and the processing circuitry uses the detected AF events to calculate AF burden (step 1006). The processing circuitry outputs a graphical presentation of the patient-initiated event data, AF burden, and the detected AF events (step 1008). The processing circuitry may be the monitor processing device 102 in FIG. 1, the processing system 106 in FIG. 1, or any other suitable processing circuitry.

At step 1002, the processing circuitry receives ECG data from a patient-portable monitoring device (e.g., monitoring device 101 worn by patient 110 as described in FIG. 1). The ECG data may be the result of continuous monitoring or periodically sampled for sufficient time to collect relevant data. At step 1004, the processing circuitry analyzes the ECG data and detects AF events. In some implementations, portions of the ECG data that do not contain AF events or VF events are discarded or otherwise disregarded. The processing circuitry may automatically detect arrhythmia events. A cardiovascular technologist (CVT) may confirm some or all of the automatically detected events. The user may select a specificity, a sensitivity, and/or a confidence level for the processing circuitry's detection process, based on a standard database or by comparing the events automatically detected by the processing circuitry to the events manually identified by the CVT. At step 1006, based on the detected AF events, the processing circuitry calculates the AF burden experienced by the patient. In some implementations, there is a minimal threshold to the amount of AF burden that is reported.

At step 1008, the processing circuitry outputs a graphical presentation of the patient-initiated event data, AF burden, and detected AF events. The graphical presentation is included in a report (e.g., report 212 or 312) that can be used by a neurologist (e.g., neurologist 108) to facilitate the identification of cardiac risks of neurological disease. The report may be formatted to show a list of the detected events (e.g., as depicted in the report section 214 or the report section 314) and a chart depicting AF burden (e.g., as depicted in the report section 216 or the report section 332). In some implementations, the report is configured to be responsive to user input (e.g., input from the neurologist 108) that may specify the time and/or date ranges of events used in the report, the types of events included in the report, the specificity and duration thresholds for including information in the report, and the configuration of the report layout. In some implementations, the neurologist may decide the number of sections included in the report and the formats of the charts included, for example, by electing to use any of the illustrative charts depicted in FIGS. 2-9.

In particular, the report output by the processing circuitry may include a graphical presentation of AF burden, detected AF events, and patient-initiated events. The graphically presented AF events include AF events having a short duration (e.g., <30 seconds, <25 seconds, <20 seconds, <15 seconds, <10 seconds or any other suitable duration). These short duration AF events can be relevant to diagnosing neurological disease even if they are less relevant to cardiac disease. For example, certain short duration AF can indicate an increased risk of stroke but not a significantly increased risk of cardiac disease (e.g., congestive heart failure). This may be because even short duration AF indicates changes in the atrium that can later cause stroke. For instance, short duration AF may be indicative of atrial cardiomyopathy, impaired atrial conduction, or other diseases of the atrial wall which could change clotting mechanisms in the heart and cause stroke.

The report output by the processing circuitry may also include a graphical presentation of patient-initiated event data. Patient-initiated events can be registered in response to a patient manually indicating an occurrence of a symptom, including symptoms that might be neurally mediated (e.g., syncope). By determining whether patient-initiated events are concurrent with detected cardiac arrhythmia, a neurologist may be able to distinguish between neurally mediated disease and cardiac disease (e.g., between neurally mediated syncope and cardiac syncope). For instance, if a patient-initiated syncope event coincides with a cardiac pause event, a level 1 ventricular fibrillation event, or any other serious cardiac arrhythmia, the report may indicate that a syncope is not primarily neurally mediated. In contrast, if a patient-initiated event indicated a syncope event that was not concurrent with cardiac arrhythmia, the report may indicate that the syncope was neurally mediated.

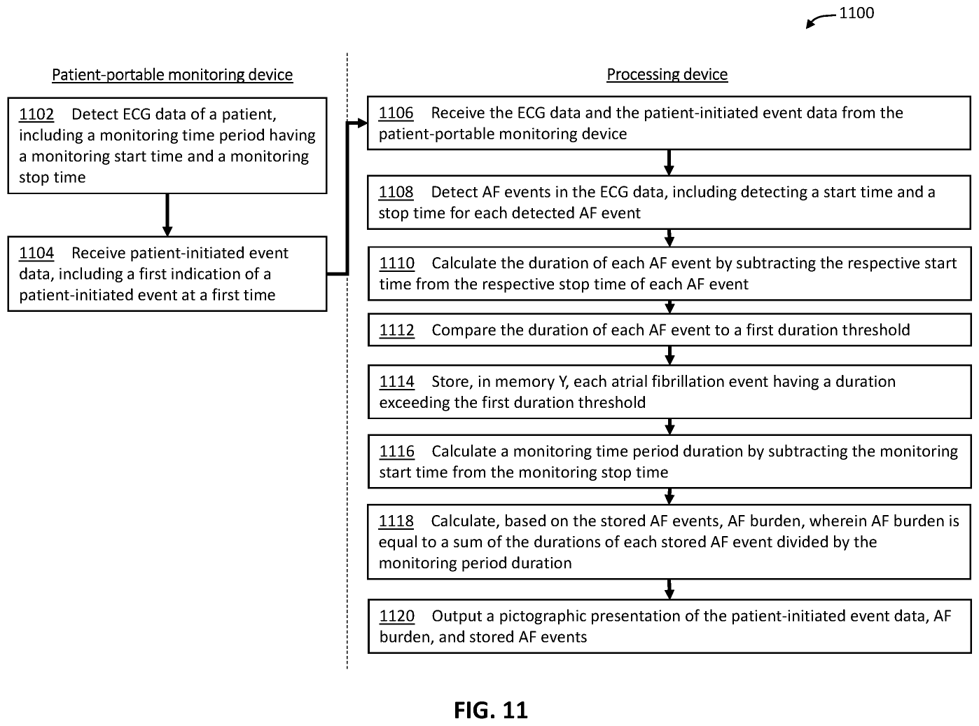

FIG. 11 shows a flow chart of a method 1100 for reporting cardiologic data according to certain implementations. The method 1100 reports atrial fibrillation (AF) events having a duration greater than a first duration threshold, which is less than 30 seconds. The method 1100 includes steps 1102, 1104, 1106, 1108, 1110, 1112, 1114, 1116, 1118, and 1120. Steps 1102 and 1104 are performed by a patient-portable monitoring device (e.g., monitoring system 109). As used herein, a “patient-portable” device includes a device that is portable and that can travel with a patient, such as wearable devices. Steps 1106, 1108, 1110, 1112, 1114, 1116, 1118, and 1120 are performed by a processing device. In some implementations, the patient portable monitoring device and the processing device are physically separate devices. In certain implementation, the entire method 1100 is performed by one device (e.g., one processor). In some implementations, the patient-portable monitoring device includes the processing device. In certain implementations, the processing device includes a server.

At step 1102, the patient-portable monitoring device detects ECG data of a patient. The ECG data includes a monitoring time period having a monitoring start time and a monitoring stop time. The monitoring start time is an indication of when ECG monitoring began or when the recording of the ECG data began. The ECG data may be collected using an ECG sensor, ECG electrodes, or any other suitable detection device. The monitoring stop time is an indication of when ECG monitoring stopped or when the recording of the ECG data stopped. The monitoring start and stop times may be stored in memory as time stamps or any other suitable data variable or structure, and in any suitable format.

At step 1104, the patient-portable monitoring device receives patient-initiated event data, including a first indication of a patient-initiated event at a first time. The indication of the patient-initiated event includes information, such as a time stamp, that specifies the first time, i.e., the time when the patient-initiated event was registered. In certain implementations, the time stamp may indicate a time span during which the patient-initiated event occurred. In some implementations, the indication of the patient-initiated event also includes a description, categorization, or type of the patient-initiated event (e.g., syncope, angina, dizziness). Such a description may be a string of text entered, or selected, by a patient. The indication of the patient-initiated event may be received using a software button, a physical button, an accelerometer, or any other suitable user interface that is accessible to the patient. In some implementations, the ECG data includes no indications of patient-initiated events, in which case step 1104 may be omitted.

At step 1106, the processing device receives ECG data and the patient-initiated event data from the patient-portable monitoring device. The ECG data and the patient-initiated event data may be wirelessly transmitted from the patient-portable monitoring device to the processing device using any suitable transmission method (e.g., via Bluetooth or other radio transmission). The ECG data may be compressed, or down-sampled, to reduce the amount of data transmitted. The processing device need not directly receive the ECG data from the patient-portable monitoring device. For example, the processing device may receive the ECG from a server which received data from the patient-portable monitoring device.

At step 1108, the processing device detects AF events in the ECG data. For each detected AF event, the processing device may determine an associated AF start time and AF stop time and store such start and stop times in non-transitory, computer-readable memory Y as an AF data structure. In addition, the processing device may also store a portion of the ECG data between the AF start time and the AF stop time in the AF data structure. The processing device may detect multiple AF events and store them in the AF data structure. Each AF event may be stored in the computer-readable memory for further analysis and processing. In some implementations, the processing device detects potential AF events in step 1108. The processing device confirms a potential AF event as an AF event only if, in step 1112, the processing device determines that the potential AF event has a duration exceeding the first duration threshold.

At step 1110, the processing device calculates the duration of each AF event by subtracting the respective start time from the respective stop time of each AF event. The processing device calculates the duration using the following formula:

[AF duration]ᵢ=[AF stop time]ᵢ−[AF start time]ᵢ

where i is an index that indicates the i^{th} AF event. The processing device may calculate the duration iteratively for all detected AF events.

At step 1112, the processing device compares the duration of each AF event to a first duration threshold. In some embodiments, the first duration threshold may be less than 30 seconds. In some implementations, the first duration threshold is 29 seconds, 28 seconds, 27 seconds, 26 seconds, 25 seconds, 24 seconds, 23 seconds, 22 seconds, 21 seconds, 20 seconds, 19 seconds, 18 seconds, 17 seconds, 16 seconds, 15 seconds, 14 seconds, 13 seconds, 12 seconds, 11 seconds, 10 seconds, 9 seconds, 8 seconds, 7 seconds, 6 seconds, 5 seconds, 4 seconds, 3 seconds, 2 seconds, 1 second, or any other suitable threshold. In certain implementations, the first duration threshold is variable and changes depending on a desired level of sensitivity or specificity. For example, the first duration threshold may be set so that the sensitivity of detection is 90% and the specificity is 95%. If the duration of an AF event does not exceed the first duration threshold, then the AF event is not stored or at least is not stored in the location where AF events having a duration greater than the first duration threshold are stored. AF events with a duration shorter than the first duration threshold may have such a high false positive rate (low specificity) that they are too unreliable to be used for medical diagnosis.

If the duration of an AF event is greater than the first duration threshold, the processing device stores the AF event in memory Y as an AF data structure at step 1114. The AF events that are stored include some AF events having a duration less than 30 seconds. In some implementations, the processing device “stores” the AF events by creating a data structure that includes pointers to each AF event having a duration greater than the first duration threshold. Storing does not necessarily require writing the underlying ECG data corresponding to the AF events (e.g., ECG data occurring between the respective start time and respective stop time of the respective AF event) to more than one memory location. In some implementations, the steps of determining the duration of each AF event (step 1110) and comparing the duration of each AF event to the first threshold (step 1112) are performed as part of detecting AF events (step 1108).

At step 1116, the processing device calculates the monitoring time period duration by subtracting the monitoring start time from the monitoring stop time. At step 1118, the processing device calculates atrial fibrillation (AF) burden based on the stored atrial fibrillation events. The AF burden is the sum of the durations of each stored atrial fibrillation event divided by the monitoring time period duration. The AF burden is calculated using the following equation:

$$[ AF burden ] = \frac{{\sum }_{i = 1}^{N} {[ AF duration ]}_{i}}{[ monitoring end time ] - [ monitoring start time ]}$$

where N is the number of stored AF events and i is the index indicating the i^{th} AF event. In an illustrative implementation, if no AF event is detected or stored in the monitoring time interval, the AF burden is determined to be zero (as though the numerator in the equation above is zero).

At step 1120, the processing device outputs a graphical presentation of the patient-initiated event data, AF burden, and stored AF events. The graphical presentation may be the graphical presentation of report 212, report 312, or any other suitable presentation.

FIG. 12 shows a flow chart of a method 1200 for reporting cardiologic data, distinguishing between short duration and long duration AF events according to certain implementations. The method 1200 can be executed by a processing device, such as processing system 106 of FIG. 1. In step 1202, the processing device receives ECG data from the patient-portable monitoring device. In some implementations, the processing device also receives patient-initiated events. The processing device need not receive the ECG data directly from the patient-portable monitoring device. For example, the processing device may receive the ECG data from a server that received data from the patient-portable monitoring device.

In step 1204, the processing device receives a first duration threshold and a second duration threshold. The second duration threshold is greater than the first duration threshold. In some implementations, the first duration threshold is 29 seconds, 28 seconds, 27 seconds, 26 seconds, 25 seconds, 24 seconds, 23 seconds, 22 seconds, 21 seconds, 20 seconds, 19 seconds, 18 seconds, 17 seconds, 16 seconds, 15 seconds, 14 seconds, 13 seconds, 12 seconds, 11 seconds, 10 seconds, 9 seconds, 8 seconds, 7 seconds, 6 seconds, 5 seconds, 4 seconds, 3 seconds, 2 seconds, 1 second, or any other suitable threshold. In certain implementations, the first duration threshold is variable and changes depending on a desired level of sensitivity and/or specificity. For example, the first duration threshold may be set so that the sensitivity is 90% and the specificity is 95%. In some implementations, the second duration threshold is 1 hour, 30 minutes, 15 minutes, 10 minutes, 5 minutes, 30 seconds, <30 seconds, or any other suitable threshold. In certain implementations, the second duration threshold is variable and changes depending on a desired level of sensitivity or specificity. For example, the first duration threshold may be set so that the sensitivity is 80% and the specificity is 97%.

In step 1206, the processing device detects AF events. Each detected AF event includes a start time and a stop time. Each AF event may also include the portion of the ECG data between the start time and the stop time. The processing device may detect multiple AF events. The processing device may store each AF event in computer-readable memory for further analysis and processing. In some implementations, the processing device detects potential AF events in step 1206, and confirms the potential AF events as AF events only if, in step 1210, the processing device determines that the potential AF events have a duration exceeding the first duration threshold.

In step 1208, the processing device calculates the duration of each AF event by subtracting the respective start time from the respective stop time of each respective AF event. The processing device calculates the duration using the following equation:

[AF duration]ᵢ=[AF stop time]ᵢ−[AF start time]ᵢ

where i is an index that indicates the i^{th} AF event. The processing device may perform the duration calculation iteratively for all detected AF events. In step 1210, the processing device compares the duration of each event to the first duration threshold. If the duration of an AF event is less than or equal to the first duration threshold, the processing device discards, or ignores, the AF event. If the duration of the AF event is greater than the first duration threshold, the processing device stores the AF event in non-transitory, computer-readable memory at step 1212. The AF events that are stored include some AF events having a duration less than 30 seconds. In some implementations, the processing device “stores” the AF events by creating a data structure that includes pointers to each AF event having a duration greater than the first duration threshold. Storing does not necessarily require writing the underlying ECG data corresponding to the AF events (e.g., ECG data occurring between the respective start time and respective stop time of the respective AF event) to more than one memory location.

In step 1214, the processing device compares the duration of each stored AF event to the second duration threshold. If the duration of the respective AF event is less than the second duration threshold, the processing device stores information representative of the respective AF event in a short duration AF database in step 1215. Again, storing does not necessarily require writing the underlying ECG data corresponding to the AF events to the short duration AF database. The short duration AF database may simply include pointers to each detected AF event that is longer than the first duration threshold and shorter than the second duration threshold.

In step 1216, the processing device outputs a graphic presentation of information representing the stored AF events. In the graphical presentation, the AF events represented in the short duration AF database are graphically distinguished from the AF events not represented in the short duration AF database. The graphical presentation may be the graphical presentation of FIG. 3, FIG. 6, or any other suitable report. The processing device can graphically distinguish the AF events represented in the short duration AF database by providing separate counts of (1) AF events represented in the short duration AF database and (2) those events not represented in the short duration AF database. For example, as discussed above in relation to FIG. 3, section 318 of report 312 includes a section 322 for information regarding “Short Duration” events (e.g., events represented in the short duration AF database) and a separate section 323 for information regarding “Long Duration” events (e.g., events not represented in the short duration AF database). Alternatively, or in addition, the processing device can perform the visual distinguishing using symbols and a legend as in FIG. 6 above.

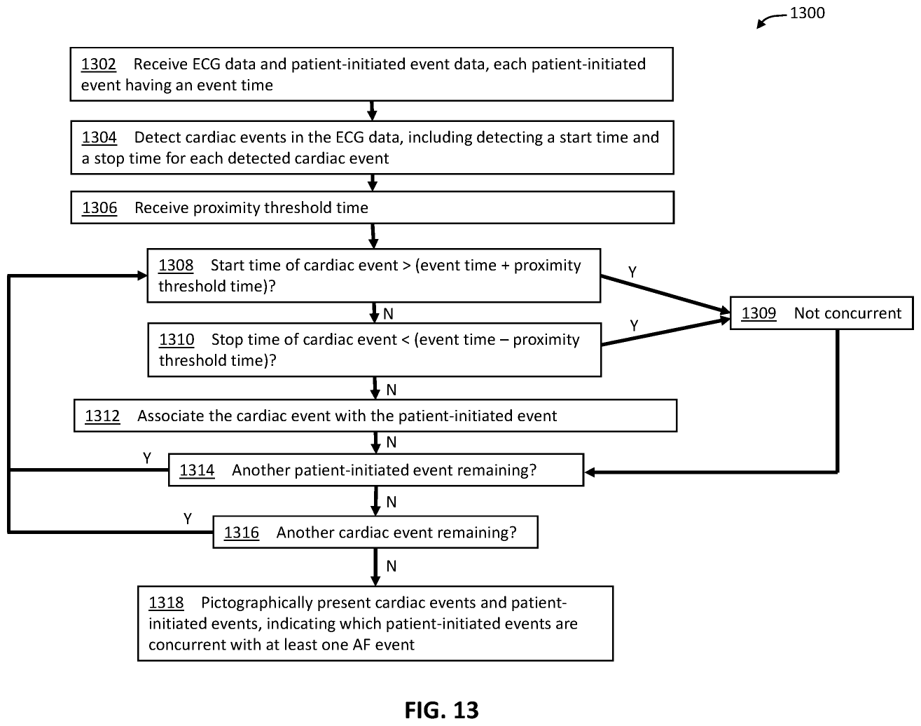

FIG. 13 shows a flow chart of a method 1300 for determining whether patient-initiated events are concurrent with automatically detected events according to certain implementations. The method 1300 can be executed by a processing device, such as processing system 106 of FIG. 1. By determining whether a patient-initiated event is concurrent with an automatically detected event (e.g., AF, cardiac pause, ventricular fibrillation), the processing device can indicate whether a patient-initiated event is linked to a cardiac disease or not. Determining concurrence, or near concurrence, of patient-initiated events and cardiac disease may distinguish between cardiac disease and neurological disease (e.g., syncope caused by cardiac pause versus neurally mediated syncope).

In step 1302, the processing device receives ECG data and patient-initiated event data. Each patient-initiated event has an event time. The event time is the time at which the patient-initiated event was registered by the patient. In step 1304, the processing device detects cardiac events in the ECG data. Each detected cardiac event includes a start time and a stop time. In step 1306, the processing device receives a proximity threshold time. The processing device uses the proximity threshold time to determine “near concurrence” as opposed to “strict concurrence.” As used herein, concurrence refers to strict concurrence and/or near concurrence. Strict concurrence occurs when two events overlap at some point in time. Near concurrence occurs when two events do not overlap at any point in time, but occur very near in time to each other. In other words, near concurrence is present when two events, though not overlapping, are within x units of time of each other, where x is the proximity threshold time, which is typically a small number. In some implementations, the proximity threshold time is 10 minutes, 5 minutes, 2 minutes, 1 minute, 30 seconds, 15 seconds, 10 seconds, 5 seconds, 4 seconds, 3 seconds, 2 seconds, 1 second, 0.5 seconds, 0.1 seconds, or any other suitable number. In some implementations, the proximity threshold time is 0 so that only strict concurrence will be detected.

In step 1308, for each detected cardiac event, the processing device evaluates the following expression (Expression 1):

[start time of cardiac event]ᵢ>(event timeⱼ+proximity threshold time)

Here, i denotes the i^{th} cardiac event and j denotes the j^{th} patient-initiated event. The processing device may compute Expression 1 for every combination of cardiac event and patient-initiated event (e.g., computed i*j times). If Expression 1 is true, then the cardiac event i began so much later than the patient-initiated event j that the cardiac event i is not concurrent with the patient-initiated event j. Thus, if Expression 1 is true, the processing device proceeds to step 1309, concluding that the cardiac event i and the patient-initiated event j are not concurrent. If Expression 1 is false, then there is a chance that the cardiac event i and the patient-initiated event j are concurrent. To determine whether there is concurrence, the processing device examines the stop time of the cardiac event i using the following expression (Expression 2):

[stop time of cardiac event]ᵢ<(event timeⱼ−proximity threshold time)

If Expression 2 is true, then the cardiac event i ended so much earlier than the patient-initiated event j that the cardiac event i could not have been concurrent with the patient-initiated event j. Thus, if Expression 2 is true, the processing device proceeds to step 1309, concluding that the cardiac event i and the patient-initiated event j are not concurrent. If Expression 1 and Expression 2 are both false, then the cardiac event i and the patient-initiated event j are concurrent (either strictly concurrent or nearly concurrent), and the processing device proceeds to step 1312. The skilled person would appreciate that the processing device can evaluate Expression 1 and Expression 2 in reverse order or even simultaneously. In any event, only if both Expression 1 and Expression 2 are false can the cardiac event i and the patient-initiated event j be concurrent. If either Expression 1 or Expression 2 are true, then the cardiac event i and the patient-initiated event j cannot be concurrent. In Expression 1 and Expression 2, later times are represented by higher values such that (event time+x) means x units of time after the event time. Analogously, (event time−x) means x units of time before the event time.

In step 1312, the processing device associates cardiac event i with the patient-initiated event j if the processing device finds both Expression 1 and Expression 2 to be false. The processing device may associate cardiac event i with the patient-initiated event j by creating a data structure (or adding an entry to an existing data structure) that includes pointers to the cardiac event i and the concurrent patient-initiated event j. After the association in step 1312, or the determination of no concurrence in step 1309, the processing device proceeds to step 1314. At step 1314, the processing device determines whether another patient-initiated event remains to be evaluated against the cardiac event i. If another patient-initiated event remains (e.g., patient-initiated event j+1), then the processing device proceeds to step 1308 and repeats the concurrence analysis (e.g., steps 1308 and 1310). If no more patient-initiated events remain, the processing device proceeds to step 1316. At step 1316, the processing device determines whether any other cardiac events remain. If one or more cardiac events remain, the processing device repeats the concurrence analysis for the next cardiac event (e.g., cardiac event i+1). If no additional cardiac events remain, the processing device proceeds to step 1318. At step 1318, the processing device graphically presents the cardiac events and patient-initiated events. The graphical presentation indicates which patient-initiated events are concurrent with cardiac events. The processing device may provide the graphical presentation in a format similar to FIG. 7 or in any other suitable format.

To provide an overall understanding, certain illustrative implementations have been described. However, it will be understood by one of ordinary skill in the art that the systems, methods, and illustrative graphical presentations described herein may be adapted and modified as is appropriate for the application being addressed, and may be employed in other suitable applications, and that such other additions and modifications will not depart from the scope of the present disclosure. Although atrial fibrillation events are primarily discussed throughout this disclosure, the methods disclosed herein, including the methods of FIGS. 10, 11, 12, and 13, can be applied to any cardiac arrhythmia event, including tachycardia, bradycardia, cardiac pause, ventricular fibrillation, or any other cardiac arrhythmia.

What is claimed is:

1. A system for reporting cardiologic data comprising:
a patient-portable monitoring device configured to:
detect electrocardiogram (ECG) data of a patient and patient-initiated event data, the ECG data including a monitoring time period comprising a monitoring start time and a monitoring stop time;
circuitry configured to:
receive the ECG data and the patient-initiated event data from the patient-portable monitoring device;
detect atrial fibrillation (AF) events in the ECG data, wherein detecting AF events includes detecting a start time and a stop time for each detected AF event;
calculate the duration of each AF event by subtracting the respective start time from the respective stop time of each AF event;
compare the duration of each AF event to a first duration threshold;
store each AF event having a duration exceeding the first duration threshold;
calculate a monitoring time period duration by subtracting the monitoring start time from the monitoring stop time;
calculate, based on the stored AF events, AF burden for the monitoring time period, wherein the AF burden for the monitoring time period is equal to a sum of the durations of each stored AF event occurring during the monitoring time period divided by the monitoring time period duration; and
output a graphical presentation of the patient-initiated event data, AF burden, and stored AF events;
wherein the first duration threshold is less than 30 seconds.

2. The system of claim 1, wherein the circuitry is further configured to exclude ECG traces from the graphical presentation.

3. The system of claim 2, wherein the monitoring time period is a first monitoring time period, and wherein the ECG data includes a second monitoring time period occurring after the first monitoring time period and comprising a second monitoring start time and a second monitoring stop time;
wherein the circuitry is further configured to:
calculate the second monitoring time period duration by subtracting the second monitoring start time from the second monitoring stop time, and
calculate, based on the stored AF events, AF burden for the second monitoring time period, wherein the AF burden for the second monitoring time period is equal to a sum of the durations of each stored AF event occurring during the second monitoring time period divided by the second monitoring time period duration; and
wherein outputting the graphical presentation of AF burden comprises graphically presenting a plot of AF burden over time, the plot including:
a first axis representing time,
a second axis representing a level of AF burden,
a graphical indication of the AF burden for the first monitoring time period, and
a graphical indication of the AF burden of the second monitoring time period.

4. The system of claim 3, wherein the second axis is scaled logarithmically.

5. The system of claim 2, wherein outputting a pictographic presentation of the AF burden comprises outputting a percentage representing the fraction of time spent in AF in a monitoring time period.

6. The system of claim 2, wherein outputting a graphical presentation of the AF events comprises outputting a count of AF events.

7. The system of claim 2, wherein the circuitry is further configured to present the respective duration of each of the graphically presented AF events.

8. The system of claim 7, wherein the circuitry is further configured to graphically present a plot of the duration of each of the presented AF events over time.

9. The system of claim 2, wherein outputting a graphical presentation of the patient-initiated events comprises outputting a count of the patient-initiated events.

10. The system of claim 2, wherein the circuitry is further configured to:
detect cardiac pause events in the ECG data; and
output a graphical presentation of the pause events.

11. The system of claim 2, wherein outputting a graphical presentation of the pause events comprises outputting a count of the pause events.

12. The system of claim 10, wherein the circuitry is further configured to output the patient-initiated event data, AF events, and pause events on a common time scale.

13. The system of claim 10, wherein the circuitry is further configured to:
detect ventricular fibrillation events in the ECG data;
output a graphical presentation of the ventricular fibrillation events;
determine a severity score of the detected ventricular fibrillation events;
compare the respective severity score of each of the detected ventricular fibrillation events to a severity threshold; and
exclude, from the graphical presentation, each ventricular fibrillation event having a severity score less than the severity threshold.

14. The system of claim 1, wherein the circuitry is further configured to:
compare the duration of each AF event to a second duration threshold, wherein the second duration threshold is greater than the first duration threshold; and
store a representation of each AF event having a duration less than the second duration threshold in a short duration AF database.

15. The system of claim 14, wherein the second duration threshold is 30 seconds.

16. The system of claim 14, wherein outputting the graphical presentation comprises visually distinguishing on the graphical presentation the AF events stored in the short duration AF database from those AF events have a duration exceeding the first duration threshold and not represented in the short duration AF database.

17. A method for reporting cardiologic data comprising:
receiving ECG data and patient-initiated event data from the patient-portable monitoring device, the ECG data including a monitoring time period comprising a monitoring start time and a monitoring stop time, the patient-initiated event data including a first patient-initiated event at a first time;
detecting atrial fibrillation (AF) events in the ECG data, wherein detecting AF events includes detecting an AF start time and an AF stop time for each detected AF event;
calculating the duration of each AF event by subtracting the respective AF start time from the respective AF stop time of each AF event;
comparing the duration of each AF event to a first duration threshold;
storing each AF event having a duration exceeding the first duration threshold;
calculating a monitoring time period duration by subtracting the monitoring start time from the monitoring stop time;
calculating, based on the stored AF events, AF burden for the monitoring time period, wherein the AF burden for the monitoring time period is equal to a sum of the durations of each stored AF event occurring during the monitoring time period divided by the monitoring time period duration; and
outputting a graphical presentation of the patient-initiated event data, AF burden, and stored AF events;
wherein the first duration threshold is less than 30 seconds.

18. The method of claim 17, further comprising excluding ECG traces from the graphical presentation.

19. The method of claim 18, wherein the monitoring time period is a first monitoring time period, and wherein the ECG data includes a second monitoring time period occurring after the first monitoring time period and comprising a second monitoring start time and a second monitoring stop time;
wherein the method further comprises:
calculating the second monitoring time period duration by subtracting the second monitoring start time from the second monitoring stop time, and
calculating, based on the stored AF events, AF burden for the second monitoring time period, wherein the AF burden for the second monitoring time period is equal to a sum of the durations of each stored AF event occurring during the second monitoring time period divided by the second monitoring time period duration; and
wherein outputting the graphical presentation of AF burden comprises graphically presenting a plot of AF burden over time, the plot including:
a first axis representing time,
a second axis representing a level of AF burden,
a graphical indication of the AF burden for the first monitoring time period, and a graphical indication of the AF burden of the second monitoring time period.

20. The method of claim 19, wherein the second axis is a logarithmic scale.

21. The method of claim 17, further comprising
comparing the duration of each AF event to a second duration threshold, wherein the second duration threshold is greater than the first duration threshold;
storing a representation of each AF event having a duration less than the second duration threshold in a short duration AF database; and
visually distinguishing the AF events represented in the short duration AF database from AF events not represented in the short duration AF database.

22. The method of claim 21, further comprising:
detecting cardiac pause events in the ECG data; and
outputting a graphical presentation of the pause events.

23. The method of claim 22, further comprising:
receiving a proximity threshold time;
for each pause event, determining whether a start time of the pause event is greater than the sum of the first time of the first patient-initiated event and the proximity threshold time;
if the start time of the pause is event is not greater than the sum of the first time and the proximity threshold time, determining whether an end time of the pause event is less than the first time minus the proximity threshold time; and
if the start time of the pause is event is not greater than the sum of the first time and the proximity threshold time, and if the end time of the pause event is not less than the first time minus proximity threshold time, indicating in the graphical presentation that the patient-initiated event and the pause event are concurrent.
